(19) United States
(12) Patent Application Publication
(10) Pub. No.: US 2017/0265920 A1
(43) Pub. Date: Sep. 21, 2017

(54) Device for supplying fluid substances in the body of a patient

(71) Applicant: TECRES S.P.A., Sommacampagna (Verona) (IT)

(72) Inventors: Giovanni FACCIOLI, Monzambano (IT); Renzo SOFFIATTI, Nogara (IT)

(21) Appl. No.: 15/504,942

(22) PCT Filed: Jul. 30, 2015
(86) PCT No.: PCT/IB2015/055786

§ 371 Date: Feb. 17, 2017

(30) Foreign Application Priority Data
Aug. 20, 2014 (IT) VR2014A000214

(51) Int. Cl. A61B 17/88

(52) U.S. Cl. CPC A61B 17/8816; A61B 17/8822; A61B 17/8825; A61B 2090/064

(57) ABSTRACT

The present invention regards a device for supplying fluid substances in the body of a patient comprising a first tubular element, a second tubular element as well as at least one component for the angular movement or articulation of the first tubular element with respect to the second tubular element around an articulation axis (z-z).

### TECHNICAL FIELD OF THE INVENTION

The present invention regards a device for supplying fluid substances in the body of a patient, for example for supplying or feeding a bone cement into a bone cavity, such as a patient's vertebra.

### STATE OF THE PRIOR ART

Up to now, many devices have been proposed for supplying bone cement in the vertebrae of patients.

The international application WO2004057280A1, on behalf of the applicant of the present patent application, teaches a surgical device for injecting bone cement in a bone cavity of a patient; such device comprises a hollow elongated element to be partially inserted in the bone cavity, a connector component in communication with a bone cement source and rotatably mounted on the hollow elongated element.

The United States patent application US20040260303 A1 instead teaches a device comprising a first element connected to a bone cement syringe or container and a second element pivoted to the first and connected to a needle to be inserted in a bone cavity of a patient.

Both above-indicated solutions, if on one hand ensure a certain relative mobility of the respective components, even during the injection of the bone cement, in any case have several structural constraints, which partly limit the freedom of action of the doctor or operator, as well as the adaptability to different operation conditions. Moreover, with such devices, the doctor or the operator who is executing the injection cannot—unless another doctor or operator is assisting—move away from the operation area, without interrupting the injection.

WO2006023430A2 and US20040122438 teach connectors according to the state of the technique.

### SUMMARY OF THE INVENTION

One object of the present invention is to provide a new device for supplying fluid substances in the body of a patient.

Another object of the present invention is to provide a device for supplying fluid substances in the body of a patient that ensures good adaptability and versatility.

Another object of the present invention is to provide a device for supplying fluid substances in the body of a patient due to which the doctor can also move away from the operation area, without interrupting the injection.

In accordance with one aspect of the invention, a device according to the present invention is provided. The present specification refers to preferred and advantageous embodiments of the invention.

### BRIEF DESCRIPTION OF THE DRAWINGS

Other characteristics and advantages of the invention will be clearly seen in the description of embodiments of a device, illustrated by way of example in the enclosed drawings in which:

FIG. 1 is a slightly top perspective view of a device according to the present invention;

FIG. 2 is an exploded view of the device of FIG. 1;

FIG. 3 is a slightly top perspective view of the device of FIG. 1 provided with a first component of connector or connection to a syringe;

FIG. 4 is a slightly top perspective view of a unit for supplying a fluid substance according to the present invention provided with the device of FIG. 1;

FIG. 5 is a slightly top perspective view of a first tubular element of a device according to the present invention;

FIGS. 6 and 7 are side views with parts in section of the first tubular element of FIG. 5;

FIG. 8 is a sectional view along the line VIII-VIII of FIG. 7;

FIG. 9 is a slightly top perspective view of a second tubular element of a device according to the present invention;

FIG. 10 is a side view of the second tubular element of FIG. 9;

FIGS. 11 and 12 are sectional views of the second tubular element of FIG. 9;

FIGS. 13 and 14 are slightly top perspective views, from respective sides, of a cap component of a device according to the present invention;

FIGS. 15, 16 and 17 are respectively top, bottom and side views of the cap component of FIG. 13;

FIGS. 18 and 19 are sectional views along the lines, respectively XVIII-XVIII and

XIX-XIX of the cap component of FIG. 13;

FIG. 20 is a slightly top perspective view of another device according to the present invention;

FIG. 21 is an exploded view of the device of FIG. 20;

FIGS. 22 and 23 are respectively lateral and front views of the device of FIG. 20;

FIGS. 24 and 25 are sectional views respectively along the lines XXIV-XXIV and XXV-XXV of the device of FIG. 22;

FIG. 26 is a slightly top perspective view of a unit for supplying a fluid substance according to the present invention provided with the device of FIG. 20;

FIG. 27 is a slightly top and exploded perspective view of another device according to the present invention; and

FIGS. 28 and 29 illustrate respective steps of use of a device according to the present invention.

In the set of drawings, equivalent parts or components are marked with the same reference numbers.

### DETAILED DESCRIPTION OF THE INVENTION

With reference to figures from 1 to 19, a device or joint 1 is illustrated for supplying fluid substances in the body of a patient according to the present invention comprising a first tubular element 2 and a second tubular element 3 that can be angularly moved with respect to each other, as will be better clarified herein below.

The first tubular element 2 has an end of connection 2a to a source of a fluid substance, such as bone cement BC, and a supply end 2b and such element 2 then defines a first flow channel 4 for conveying a fluid substance from the end of connection 2a to the supply end 2b. In addition, the first tubular element 2 has at least one first segment 2c that extends along or around a first longitudinal axis x-x, if desired a longitudinal symmetry axis of the first segment 2c, such first segment 2c delimits at least part of the first flow channel 4.

The second tubular element 3 instead has an inlet end 3a for the fluid substance as well as an outlet end 3b for feeding the fluid substance outside the device 1 and if desired within or towards a needle to be inserted in the body of a patient, for example in a bone cavity thereof, as will be better described herein below. The second tubular element 3 defines a second flow channel 5 in fluid communication with the first flow channel 4 and intended to convey a fluid substance from the inlet end 3a to the outlet end 3b. The second tubular element 3 also has at least one first section 3c extended along or around a second longitudinal axis y-y, if desired a longitudinal symmetry axis of the first section 3c, such first section 3c at least partly delimiting the second flow channel 5.

The first tubular element 2, moreover, is angularly movable with respect to the second tubular element 3 around an articulation axis z-z transverse or orthogonal to the first segment 2c of the first tubular element 2 and, if desired, to the first section 3c. According to the embodiment illustrated in the figures, the articulation axis z-z is substantially orthogonal both to the first longitudinal axis x-x and to the second longitudinal axis y-y.

Advantageously but not necessarily, one or more articulation or angular movement components 2d, 3d are provided for the angular movement or articulation of the first tubular element 2 with respect to the second tubular element 3 around an articulation axis z-z, such articulation or movement component/s 2d, 3d extending or developing transverse or orthogonal to the first segment 2c of the first tubular element 2 and extends/extend along the articulation axis z-z so as to offset the first segment 2c of the first tubular element 2 and the first section 3 c of the second tubular element 3 in the direction of the articulation axis z-z. If desired, the articulation or movement component/components 2d, 3d is/are also transverse or orthogonal to the first section 3c of the second tubular element 3.

This offset is such to ensure the non-alignment of the first segment 2c of the first tubular element 2 with respect to the first section 3c of the first tubular section 3, so as to ensure the rotation or angular movement of the first tubular element 2 around the articulation axis z-z without the second tubular element 3 interfering with or obstructing such movement.

If desired, the first segment 2c of the first tubular element 2 is articulated with respect to the second tubular element 3 in a manner such to define an articulation plane AP orthogonal to the articulation axis z-z and passing through the first longitudinal axis x-x. In such case, the first section 3c of the second tubular element 3 is offset with respect to the articulation plane PA, more particularly it can be offset by a distance at least or, preferably, substantially equal to the transverse size of the first segment 2c of the first tubular element 2.

In substance, due to the articulation or angular movement components 2d, 3d, the first tubular element 2 is rotatably and projectingly supported, along the direction of the articulation axis z-z, by the second tubular element 3.

Advantageously, the inlet end 3a of the second tubular element 3 is articulated to the supply end 2b of the first tubular element 2 around the articulation axis z-z.

As will be understood, this can be obtained by directly pivoting or articulating, i.e. without intermediate components, the inlet end 3a to the supply end 2b, or by providing or arranging an intermediate component on one side constrained to one of the tubular elements 2 or 3 and on the other side rotatably anchored or articulated to the other of the tubular elements 3 or 2.

Preferably, angular movement or articulation component/s comprises/comprise a shank component 3d of one from among the first tubular element 2 and the second tubular element 3 and defining the inlet end 3a or the supply end 2b, as well as an annular or cradle component 2d of the other from among the second tubular element 3 and the first tubular element 2 and defining the other from among the supply end 2b or the inlet end 3a. The annular or cradle component 2d then delimits a seat 6 for the housing and articulated connection of the shank component 3d around the articulation axis z-z, so as to allow the articulation of the supply end 2b with respect to the inlet end 3a around the articulation axis z-z.

If desired, the shank component 3d is extended transversely or orthogonally from the first segment 2c of the first tubular element 2 or from the first section 3c of the second tubular element 3, so as to respectively define a second segment of the first tubular element 2 or a second section of the second tubular element 3.

In such case, the first tubular element 2 or the second tubular element 3 is substantially configured as an elbow, with one arm of the elbow comprising the first segment 2c or the first section 3c, while the other arm of the elbow includes the shank component 3d.

The annular or cradle component 2d like the shank component 3d is extended along or around the articulation axis z-z, which can constitute a longitudinal axis, if desired a longitudinal symmetry axis of the annular or cradle component 2d and/or of the shank component 3d.

Preferably, the first tubular element 2 is formed in a single piece, if desired via molding. For such purpose and with reference to the non-limiting embodiment illustrated in the figures, from the end of connection 2a to the supply end 2b, the first tubular element 2 comprises the first segment 2c and then the annular or cradle component 2d, which for example can include a substantially cylindrical segment extended transversely or orthogonally starting from the terminal end of the first segment 2c and internally defines the seat 6 for housing and articulated connection. The substantially cylindrical segment 2d and the housing and articulated connection seat 6 have longitudinal symmetry axis, preferably orthogonal to the first longitudinal axis x-x, which during use coincides with the articulation axis z-z. If desired, the first tubular element 2 comprises a sleeve component 2e defining the end of connection 2a, then the first segment 2c, for example with transverse external diameter or section greater than the sleeve component 2e and then the annular or cradle component 2d.

With one such structure, the first flow channel 4 is at least partly delimited by the first segment 2c and, if provided, partly by the sleeve component 2e and then has an inflow opening 4a at the end of connection 2a, delimited by the first segment 2c or, if provided, by the sleeve component 2e, as well as a terminal opening 4b, at the supply end 2b, delimited by the first segment 2c. At the terminal opening 4b, the first flow channel 4 opens into the housing and articulated connection seat 6, for conveying fluid, as will be better described herein below, into the second flow channel 5. If desired, the first flow channel 4 has substantially constant diameter or section for the entire extension thereof and can be substantially straight or rectilinear. The housing and articulated connection seat 6 instead preferably has cylindrical configuration with substantially constant section, if desired open at both ends 6a, 6b thereof.

The first tubular element 2, or if desired and if provided the sleeve component 2e, can have means 7 for anchoring, for example an externally threaded portion or a bayonet anchoring portion, to a syringe 11 or to a first connector or connection component 10a, in turn connected to a syringe 11.

Advantageously, the second tubular element 3 is formed in a single piece, if desired via molding. For such purpose and with reference to the non-limiting embodiment illustrated in the figures, the second tubular element 3 comprises, from the inlet end 3a to the outlet end 3b, the first section 3c and the shank component 3d, the latter preferably extended transversely or orthogonally from the first section 3c and both delimiting a respective first 5a and second 5b branch, transverse or orthogonal to each other, of the second flow channel 5, preferably having diameter or section that is constant or substantially equal to each other. With regard in particular to the shank component 3d, this has an internal wall delimiting the second branch 5b of the second flow channel 5 and an external wall suitably shaped so as to be insertable within the housing and articulated connection seat 6. Starting from the external wall of the shank component 3d or from the internal surface of the cradle or annular component 2d, two or more annular ribs 3e, 3f, 3g and 3h can be extended, e.g. three or four, which are suitably spaced, for a reason that will be described herein below, and have external diameter and section such to be insertable to size within the housing and articulated connection seat 6.

In the wall of the shank component 3d, one or more through holes 5c are also formed that are intended to place in fluid communication the zone outside the shank component 3d with the second flow channel 5, more in detail with the second branch 5b of the second flow channel 5. More particularly, during use, the through hole/s 5c is/are intended to place in fluid communication the first flow channel 4 and the second flow channel 5. The through hole/s 5c can be obtained in one side of the shank component 3d delimited between two respective annular ribs 3f and 3g.

If desired, the second tubular element 3 comprises, from the inlet end 3a to the outlet end 3b, a bush component 3n, 3p defining the outlet end 3b, then the first section 3c, e.g. with external transverse section or diameter greater than the bush component 3n, 3p and then the shank component 3d. The bush component 3n, 3p can comprise a first externally tapered wall 3n and then an externally substantially cylindrical part 3p close to the first section 3c.

With one such structure, the second flow channel 5 is at least partly delimited by the first section 3c and, if provided, partly by the bush component 3n, 3p and has an outlet opening 5d, delimited by the first segment 3c or, if provided, by the bush component 3n, 3p, as well as an inlet opening 5c delimited by the shank component 5d and for example corresponding to the aforesaid hole/s 5c.

At the first section 3c or, if provided, at the bush component 3n, 3p, a needle 12 can then be connected, directly or by means of interposition of a second connector or connection component or nut 10b, such needle 12 to be inserted in the body of a patient, as will be described herein below, for example a needle 12 with bevel tip 12, which has an opening substantially lateral with respect to the longitudinal extension of the needle. To this regards, a device according to the present invention comprises means for removably fixing the outlet end 3b to a needle 12 or, if provided, a secondo connector component or nut 10b, the outlet end 3b to the second connector component 10b and the latter to a needle 12, so that it is possible to removably fix the device 1, or better the secondo tubular component 3 to the needle 12 itself. For such purpose, the means for removably fixing can include, for example, a thread or snap-fit engagement or another type of engagement, but in such a way as after having constrained the device to the needle, the second tubular component 3 and the needle 12 cannot rotate with respect to each other or mutually, unless the same components are deliberately disconnected (with interposition or with no interposition of a second connector component 10b), particularly at the end of the bone cement BC supply. Then, in such case, the needle 12 could rotate, together with the second tubular component 3, with respect to the first tubular component 2 about the articulation axis z-z only, but not about the second longitudinal axis y-y, and the rotation of the needle 12 about this axis y-y could occur only by rotating the whole unit 16, which will be better described in the following.

The second tubular component 3 can then comprise suitable engagement means or components 8, 9 due to which it is possible to connect, with rotatable engagement, the shank component 3d to the annular or cradle component 2d, such that the latter can be connected or removably connected to the shank component 3d, but angularly movable with respect thereto around the articulation axis z-z. The engagement means or components can comprise one or a pair of tabs or protuberances 8, if desired elastically yielding with free end curved so as to delimit a tooth or a shoulder 8a; the tabs or protuberances 8 are for example extended from the end of the shank component 3d far from the first section 3c, which actually corresponds to the inlet end 3a of the second tubular element 3.

The means for the engagement and rotatable connection of the shank component 3d to the annular or cradle component 2d can then for example also comprise a cap component 9 intended to engage, if desired removably, the tabs or protuberances 8 of the shank component 3d, such to rotatably anchor the shank component 3d to the annular or cradle component 2d around the articulation axis z-z. For such purpose, the cap component 9 can have a base segment 9a as well as a stem segment 9b insertable within the seat 6, but on an opposite side with respect to the end 6a of insertion of the shank component 3d, i.e. within the end 6b so as to engage the latter and maintain it inserted in position within the housing and articulated connection seat 6.

More particularly, in the base segment 9a of the cap component 9, one or more openings 9c is formed for the insertion and locking, if desired snap insertion/locking, of a respective tab or protuberance 8.

Of course, the engagement means could also be different from those described above and illustrated in the figures, e.g. one or more annular grooves formed in the housing and articulated connection seat 6 as well as one or more elastically yielding tabs extended from the shank component and intended to slidable engage one or a respective annular groove. Alternatively, the shank component 3d could be substantially inserted to size within the housing and articulated connection seat 6, but with the possibility of mutually rotating such components by means of application of a suitable force.

A device according to the present invention, therefore, in accordance with the specific non-limiting embodiment illustrated in the figures, provides for a first tubular element 2 and a second tubular element 3 connected, for example by means of insertion of the shank component 3d of the second tubular element within the housing and articulation seat 6, and then anchored in position within the same by means of engagement means 8, 9.

In addition, the device can comprise suitable sealing means for the fluid seal connection between the first flow channel and second flow channel. For such purpose, between the annular or cradle component 2d and the shank component 3d, one or more gaskets 13a, 13b, if desired O-ring, are preferably housed.

The sealing means can, for example, comprise at least two annular gaskets 13a, 13b mounted on the shank component 3d so as to delimit with the cradle or annular component 2d and with the shank component 3d a first zone of the housing and articulated connection seat 6. In this case, the first flow channel 4 opens in the first zone of the housing and articulated connection seat 6 and the at least one through hole 5c opens in the first zone.

If desired, the sealing means comprise a first gasket 13a between two respective annular ribs 3e and 3f and a second gasket 13b between two other respective annular ribs 3g and 3h. In such case, the first flow channel 4 opens into a zone of the housing and articulated connection seat 6 between the two annular ribs 3f and 3g delimiting the part of the shank component 5d in which the hole/s 5c is/are obtained, such that a fluid substance fed into the flow channel 4, crosses the latter, exits from this at the terminal opening 4b, entering into the annular band of the housing and articulated connection seat 6 delimited between the annular or cradle component 2d and the shank component 3d and is introduced into the second flow channel 5 through the hole/s 5c.

As stated above, one such device can be connected on one side, at the end of connection 2a, to a syringe 11 or to a first connector or connection component 10a, in turn connected to a syringe 11, so as to place the first flow channel 4 in fluid communication with the internal volume of the syringe 11, and on the other side, at the outlet end 3b, to a needle 12, if desired to a second connector or connection component 10b in turn connected to a needle 12, so as to place the second flow channel 5 in fluid communication with the internal gap of the needle 12, to be introduced and inserted in the body of a patient, for example in a vertebra thereof for the insertion of bone cement in such patient. The fluid substance can then be thrust from the syringe 11, into the device 1 and then into the needle 12 and into the patient, by operating on a suitable knob 13 intended to actuate a thrust means for the fluid substance into the syringe 11, such as a screw or the like 14 acting on a piston (not visible in the figures) or another similar component slidably mounted in the syringe 11. Additionally, the operator can maintain the syringe 11 at a grip 15 in which the screw 14 or the syringe 11 itself is fit.

A device according to the present invention assembled as stated above with a syringe 11 and a needle 12, constitutes a unit 16 for supplying a fluid substance according to the present invention.

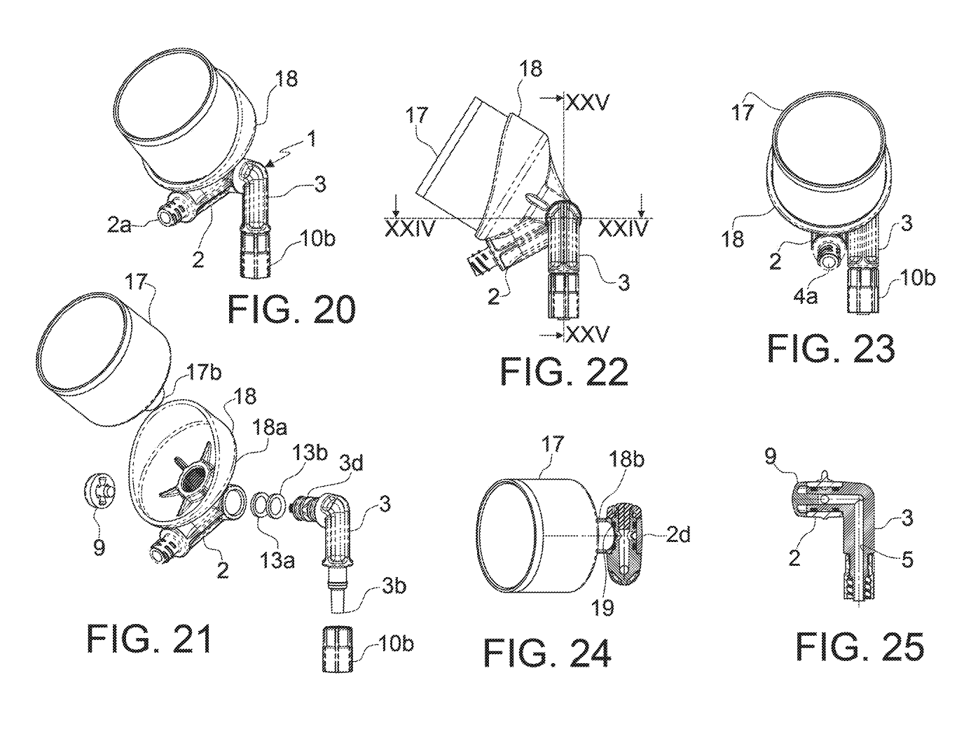

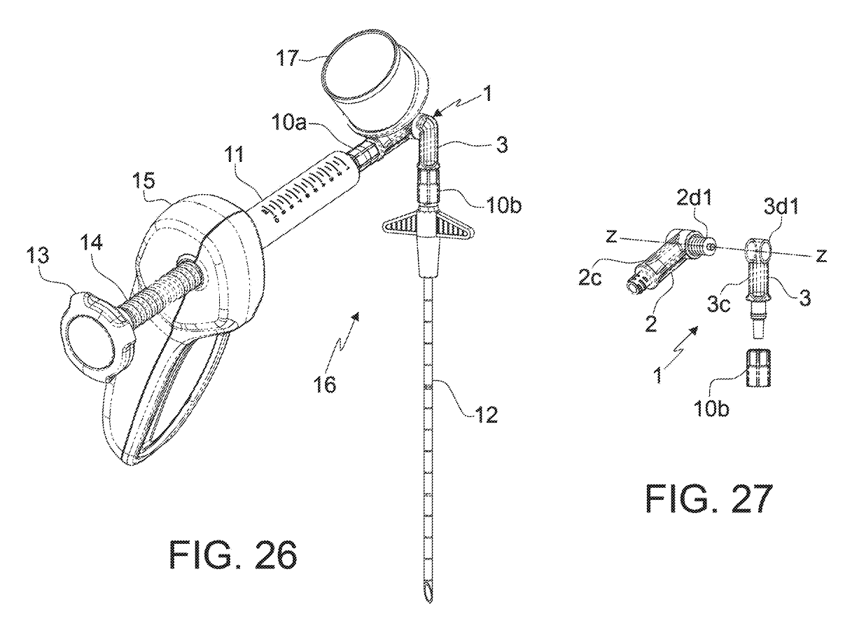

According to a variant illustrated in FIGS. 20 to 26, a device according to the present invention also provides for a pressure measurement device, such as a manometer 17 intended to measure the pressure of the fluid substance in the flow channel 4 or 5. For such purpose, the device can include a support component 18 for the manometer 17, mounted on the device, if desired connected or formed in a single piece with the first tubular element 2, if desired extended from the annular or cradle component 2d of the same. The support component 18 can have a cap 18a that during use houses part of the body 17a of the manometer 17 as well as a protuberance 18b delimiting a hole, if desired threaded, for engaging via screwing a shank 17b, if desired externally threaded, of the manometer 17. A membrane 19 is then provided, housed between the support component 18 and the first 4 or second 5 flow channel, which is suitably stressed by the fluid in the flow channel 4 or 5 and determines a corresponding pressure measurement by the manometer 17.

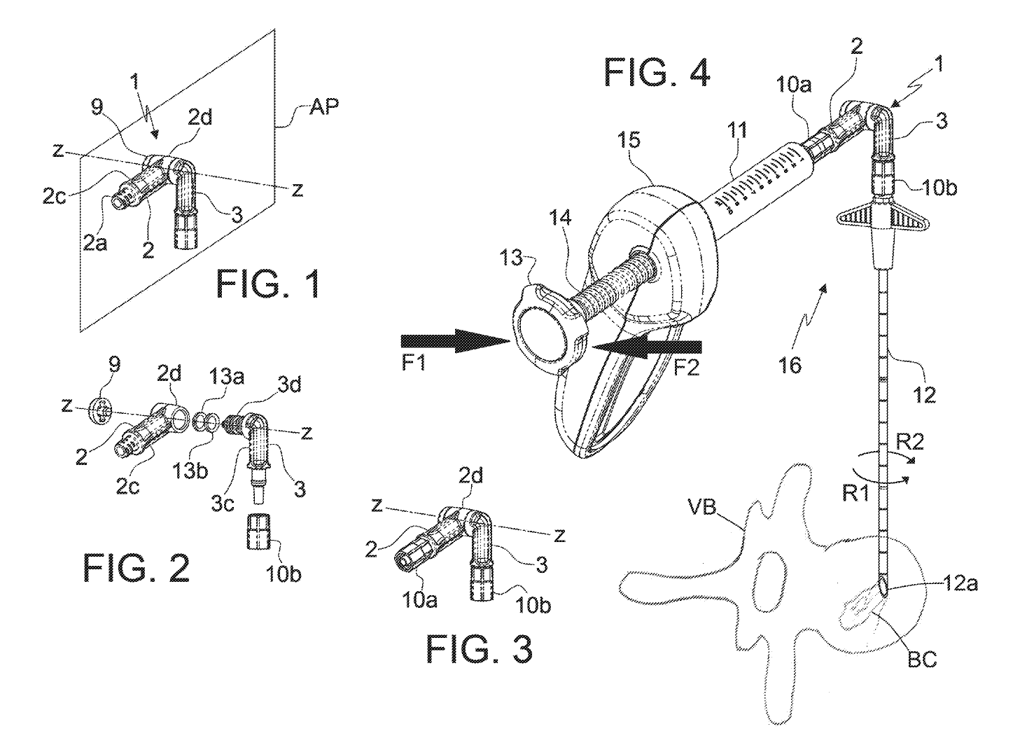

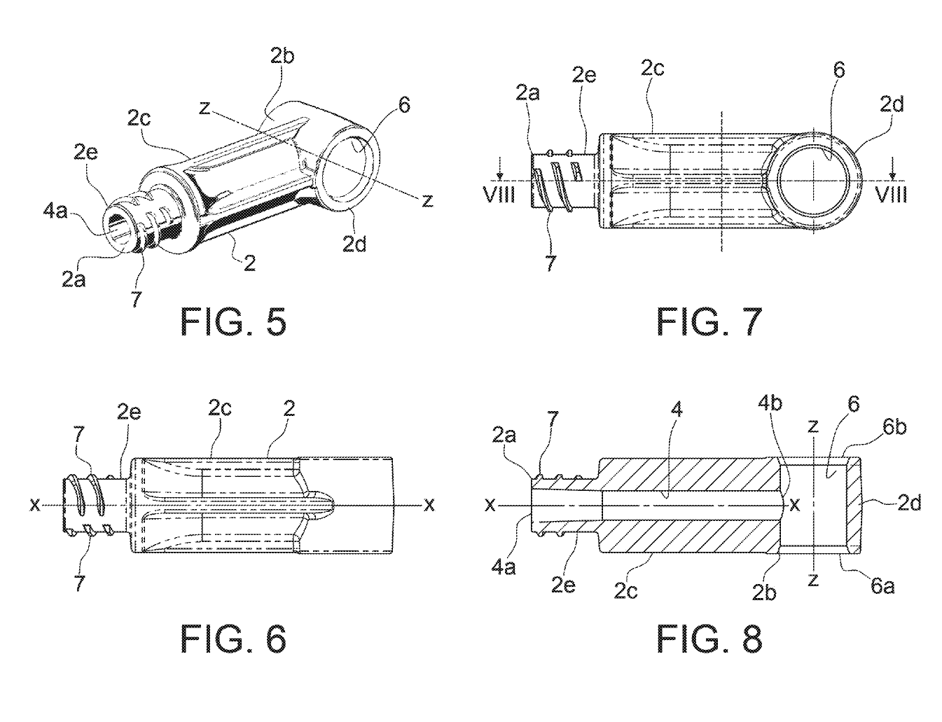

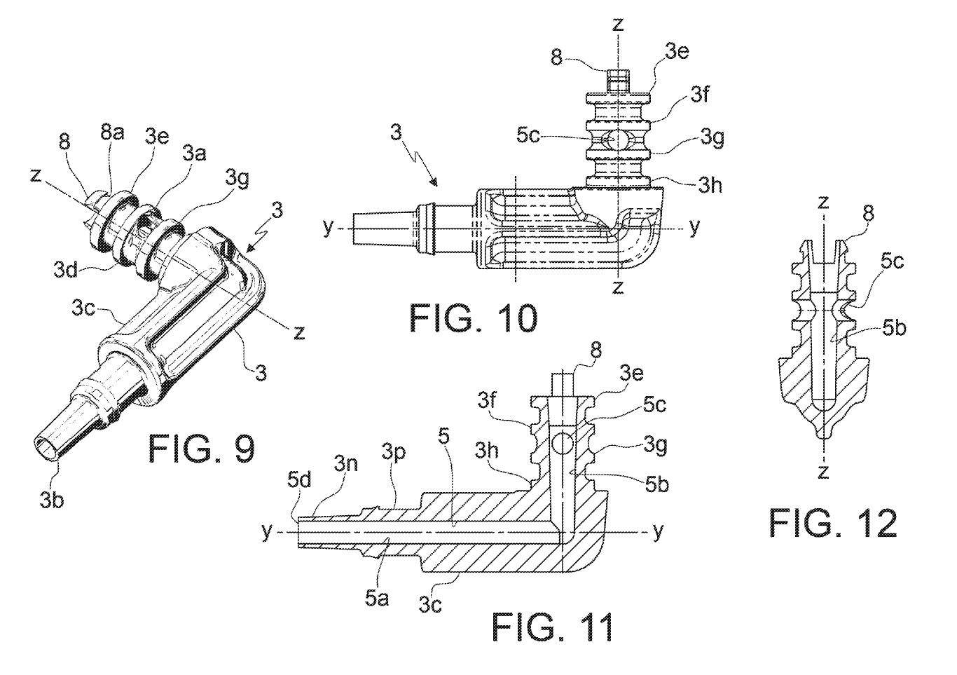

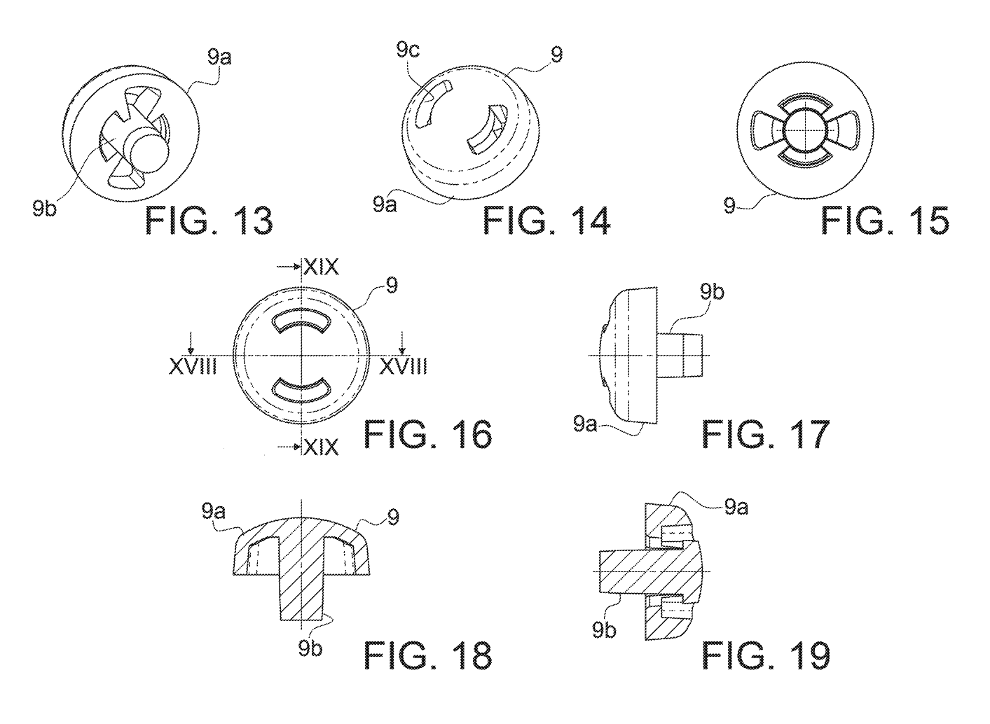

With reference now to FIG. 27, a device according to the present invention is shown that is similar to the device of FIG. 1, but in which the shank component 2d1 is part of the first tubular element 2 and is extended from the first segment 2c of the latter, while the annular or cradle component 3d1 is part of the second tubular element 3 and is extended from the first section 3c of the same.

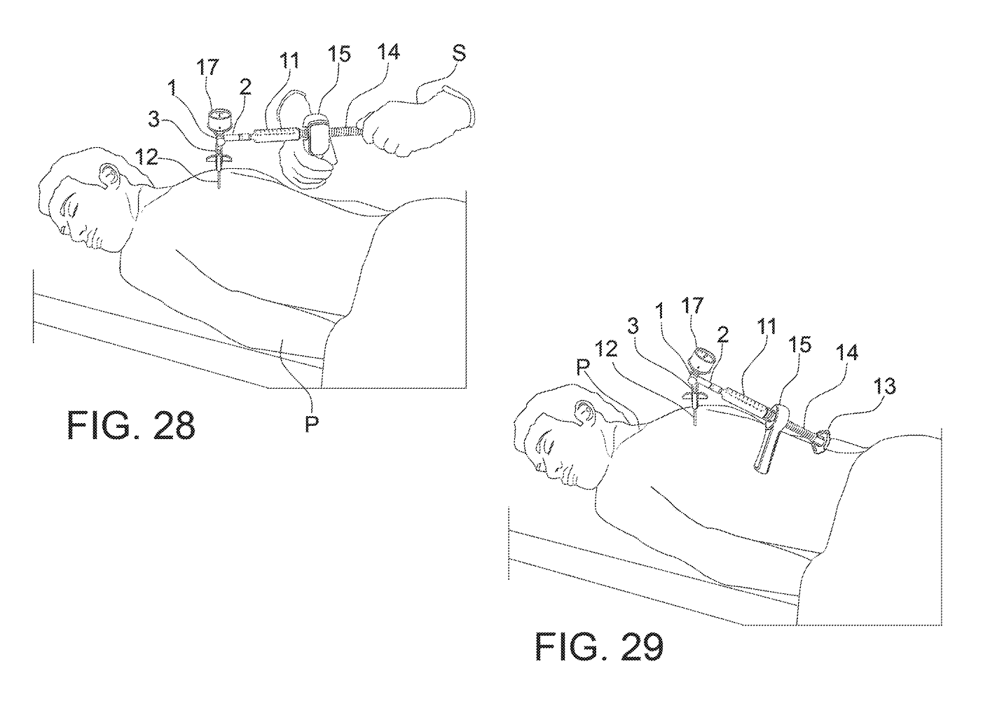

With a device according to the present invention, suitably connected with a syringe 11 and a needle 12, one inserts or introduces the needle 12 inside the body of the patient P, for example in a vertebra thereof and hence the doctor or operator S operates on the syringe 11 so as to insert the fluid substance within the body of the patient (see FIG. 28). During such step, the doctor or operator S holds the syringe 11 lifted.

If the doctor or operator S should move away from the patient P, at least temporarily leaving the device 1, so as to rest it, for example on the back of the patient P (see FIG. 29), or move the device 1, for example to bring the syringe 11 higher or lower or on the opposite side of the patient P, this could be easily attained due to the expedient according to which the first section 3 c of the second tubular element 3 is offset as stated above. Such characteristic of a device according to the present invention in fact allows obtaining an articulation or angular movement of the first tubular element 2 with respect to the second tubular element 3, without any interference or obstruction by the second tubular element. Such articulation also allows obtaining an angular movement between the two tubular elements 2 and 3 for a wide range or angular interval, e.g. 180°, if desired also 270° or even 360° around the articulation axis z-z.

In addition, with a device according to the present invention, good visibility of the operation zone can also be obtained, considering the fact that the two tubular elements 2, 3 are not actually aligned in the same articulation plane.

It should also be noted that a device according to the present invention is easy to achieve as well as assemble, considering the fact that in order to connect the two tubular elements, it is sufficient, according to the non-limiting embodiment illustrated in the drawings, to insert the shank component 3d in the housing and articulated connection seat 6, if desired with interposition of gaskets, and then engage the engaging means.

With the devices proposed up to now, instead, it is not possible to obtain a satisfactory articulation between respective components. In particular, with regard to US20040260303A1, it teaches a device with a first element and a second element pivoted to the first, but it is not clear how the same are pivoted, and in any case from what can be understood, the described structure provides for limits of articulation and pivoting between the components as well as a complex assembly thereof.

With reference instead to WO2006023430A2, first of all, such document teaches a device for supplying comprising a rotatable adaptor or connector including a first and a second tubular section, as well as a first and a second hollow housing and in which the first tubular section is mounted for rotation about a first axis with respect to the first hollow housing, whereas the second tubular section is mounted for rotation about a second axis with respect to the second hollow housing, which second axis is orthogonal to the first axis.

As it will be appreciated, such device for supplying comprises a structure much more complex and having more components than a device according to the present invention, and this clearly implies advantages both for manufacturing and for assembling the device in accordance with the present invention with respect to the solution of the prior art document now being examined.

Moreover, in the device of WO2006023430A2 the components rotate about two articulation axes and, more particularly, an articulation even with respect to the needle occurs, which, when the bone cement is inserted, could render the localization of the trim of the needle bevel tip by the operator or the surgeon very complex. On the contrary, a device according to the present invention, in accordance with a preferred variant, comprises means for removably fixing to a needle, and, owing to such an expedient, it is always possible to know, during the treatment, the arrangement of the needle and of the respective bevel tip, thereby obtaining clear advantages both of safety and treatment speed kind.

With reference to such an aspect, the needle which is preferred during the vertebroplasty or kyphoplasty is the so-called with bevel tip or with tip 12a shaped as a “flute spout”, which tip 12a makes it possible to enter with high precision into the soft tissues and the vertebral bone. This tip, owing to its substantially flat shape, acts as a guide and when it is suitable moved, it allows the doctor to reach the point in the vertebral body pre-established for inserting a substance, such as bone cement BC. Moreover, taking into consideration that the bevel tip has an opening actually lateral, it makes it possible to laterally supply cement.

If one moves or better determines an angular movement of the needle 12 and thus of the tip 12a, it is possible to supply cement to different points of a vertebral body VB. It may happen that the cement is supplied at a slit of the vertebral body VB and this event, which is not rare, represents a serious danger for the patient, since the cement, in this case, could come out the vertebral body VB, thereby causing very serious, and even lethal, damages. In such case, if a needle with bevel tip is provided, it is sufficient to rotate the needle, even by few degrees about its longitudinal axis, so as to move the cement away from the slit in the vertebral body VB, thereby actually eliminating the danger.

It is derivable from the above that the doctor should maintain a complete control of the position of the needle even referring to the position of the lateral opening of the bevel tip 12a. According to a preferred variant of the invention subject-matter of the present patent application, the device for supplying is firmly fixed or fixable to a needle 12 and therefore the doctor always knows where the tip 12a of the latter is faced, irrespectively from the manoeuvre being made and it is not possible for the tip 12a to be accidentally moved and moved independently from the device for supplying.

To this regard, in fact, should a force F1 be applied to the know 13, the unit 16 and thus, in turn, the needle 12 would be rotated about the axis y-y or about an axis parallel thereto in a direction of rotation R1, whereas should a force F2 be applied to the knob 12 in a direction opposite to the force F1, the unit 16 and thus, in turn, the needle 12 would always be rotated about the axis y-y or about an axis parallel thereto but with a direction of rotation R2 opposite to R1.

On the contrary, in accordance with the solutions of the state of the technique and particularly of WO2006023430A2, the needle is free to rotate relatively to the respective device for supplying and this since no constrain or fixing between such components is provided. This fact, as above indicated, is very dangerous, since the tip is free to randomly rotate and the doctor cannot exactly establish where the cement will come out and the doctor would need of the help of a second operator arranged to keep the needle in position. Moreover, even in this case, the supply of cement will be inaccurate and not satisfactory, since it will not be established by the doctor, but by another person or with the help of another person.

Modifications and variants of the invention are possible within the protective scope defined by the claims.

1. A device for supplying fluid substances in the body of a patient, comprising:
a first tubular element having an end of connection to a source of a fluid substance and a supply end, said first tubular element defining a first flow channel for conveying a fluid substance from said end of connection to said supply end, said first tubular element having at least one first segment extending along a first longitudinal axis (x-x);
a second tubular element having an inlet end as well as an outlet end for said fluid substance, said second tubular element defining a second flow channel in fluid communication with said first flow channel and intended to convey a fluid substance from said inlet end to said outlet end, said second tubular element having at least one first section extending along a second longitudinal axis (y-y);
further comprising at least one articulation or angular movement component for articulating or angularly moving said first tubular element with respect to said second tubular element around an articulation axis (z-z), said articulation axis being transverse or orthogonal to said first segment of said first tubular element, said at least one articulation or angular movement component extending along said articulation axis (z-z) so as to offset said first segment of said first tubular element and said first section of said second tubular element in the direction of said articulation axis (z-z), and wherein said at least one articulation or angular movement component comprises:
a shank component of one from among said first tubular element and said second tubular element, said shank component defining said inlet end or said supply end, as well as
a cradle or annular component of the other from among said second tubular element and said first tubular element, said cradle or annular component defining the other from among said supply end or said inlet end,
said cradle or annular component delimiting a seat for housing and articulated connection of said shank component around said articulation axis (z-z), thereby allowing the articulation of said supply end with respect to said inlet end around said articulation axis (z-z), and
wherein said first tubular element or said second tubular element is substantially configured as an elbow, one arm of said elbow comprising said first section or said first segment, whereas the other arm of said elbow includes said shank component,
said device comprising engagement means for connecting, with rotatable engagement, said shank component with said cradle or annular component.

2. The device according to claim 1, wherein said at least one articulation or angular movement component is transverse or orthogonal to said at least one first segment of said first tubular element and to said first section of said second tubular element.

3. The device according to claim 1, wherein or said first segment of said first tubular element is articulated with respect to said second tubular element in a manner so as to define an articulation plane (PA) orthogonal to said articulation axis (z-z) and passing through said first longitudinal axis (x-x), and in that said first section of said second tubular element is offset with respect to said articulation plane (PA).

4. The device according to claim 3, wherein only one among said first tubular element or said second tubular element is substantially configured as an elbow.

5. The device according to claim 1, wherein said inlet end of said second tubular element is articulated to said supply end of said first tubular element around said articulation axis (z-z).

6. The device according to claim 1, comprising means for removably fixing said outlet end to a needle.

7. The device according to claim 1, wherein from said end of connection to said supply end, said first tubular element comprises said first segment and then said cradle or annular component, said cradle or annular component including a substantially cylindrical segment extended transversely or orthogonally starting from the terminal end of said first segment and internally defining said seat for housing and articulated connection.

8. The device according to claim 7, wherein said substantially cylindrical segment and said seat for housing and articulated connection have longitudinal symmetry axis coinciding with said articulation axis (z-z).

9. The device according to claim 1, wherein said second tubular element comprises, from said inlet end to said outlet end, said first section and said shank component, said shank component extending transversely or orthogonally from the first section and both delimiting a respective first and second branch, transverse or orthogonal to each other, of said second flow channel.

10. The device according to claim 9, wherein said shank component has an internal wall delimiting said second branch of said second flow channel and an external wall shaped so as to be insertable within said seat for housing and articulated connection, and in that in the wall of said shank component at least one through hole is formed intended to place in fluid communication the zone outside said shank component and said second flow channel.

11. The device according to claim 1, wherein at least one from among said first tubular element (2) and said second tubular element (3) is formed in a single piece.

12. (canceled)

13. The device according to claim 1, wherein said engagement means comprise at least one tab or protuberance extending from said shank component and a cap component intended to engage said at least one tab or protuberance, so as to rotatably constrain said shank component to said cradle or annular component about said articulation axis (z-z).

14. The device according to claim 13, wherein said cap component has a base segment as well as a stem segment insertable within said seat for housing and articulated connection, but on an opposite side with respect to the end of insertion in said seat of said shank component, so as to engage said shank component and maintain it inserted in position within said seat for housing and articulated connection.

15. The device according to claim 1, comprising sealing means for the fluid seal connection between said first flow channel and said second flow channel.

16. The device according to claim 10, wherein said sealing means comprise at least two annular gaskets mounted on said shank component so as to delimit with said cradle or annular component and with said shank component a first zone of said seat for housing and articulated connection, in that said first flow channel opens in said first zone of said seat for housing and articulated connection and in that said at least one through hole opens in said first zone.

17. The device according to claim 16, further comprising at least four annular ribs suitably spaced from one another and each extending from the external wall of said shank component or from the internal surface of said cradle or annular component, and in that each gasket is located between two respective annular ribs of said at least four annular ribs.

18. The device according to claim 1, further comprising a pressure gauge component intended to measure the pressure of the fluid in said first and/or in said second flow channel.

19. The device according to claim 18, wherein said pressure gauge component comprises a manometer and in that said device includes a support component for said manometer extending from said cradle or annular component.

20. The device according to claim 19, further comprising a membrane housed between said support component and said first or second flow channel, said membrane being arranged to be stressed by the fluid flowing in said first or second flow channel, thereby determining a corresponding pressure measurement by said manometer.

21. A unit for supplying a fluid substance comprising at least one device according to claim 1, at least one syringe connected to said end of connection as well as a needle connected to said outlet end.

22. The unit for supplying according to claim 21, wherein said outlet end is removably fixed to said needle.

23. The device according to claim 1, wherein said first flow channel has substantially constant diameter or section for the entire extension thereof and is substantially straight or rectilinear.

24. The device according to claim 1, wherein said shank component is part of said first tubular element and is extended from said first segment of the latter, while said annular or cradle component is part of said second tubular element and is extended from said first section of the same.
